(19) United States
(12) Patent Application Publication
(10) Pub. No.: US 2022/0207370 A1
(43) Pub. Date: Jun. 30, 2022

(54) INFERRING DEVICE, TRAINING DEVICE, INFERRING METHOD, AND TRAINING METHOD

(71) Applicant: Preferred Networks, Inc., Tokyo (JP)

(72) Inventor: Daisuke MOTOKI, Tokyo-to (JP)

(73) Assignee: Preferred Networks, Inc., Tokyo (JP)

(21) Appl. No.: 17/698,950

(22) Filed: Mar. 18, 2022

Related U.S. Application Data
(63) Continuation of application No. PCT/JP2020/035307, filed on Sep. 17, 2020.

(30) Foreign Application Priority Data
Sep. 20, 2019 (JP) 2019-172034

(51) Int. Cl. G06N 3/08; G06N 5/04

(52) U.S. Cl. CPC G06N 3/08; G06N 5/04

(57) ABSTRACT

An inferring device includes one or more memories and one or more processors. The one or more processors input a vector relating to an atom into a first network which extracts a feature of the atom in a latent space from the vector relating to the atom, and infer the feature of the atom in the latent space through the first network.

### FIELD

This disclosure relates to an inferring device, a training device, an inferring method, and a training method.

### BACKGROUND

Quantum chemical calculation such as first-principles calculation of DFT (Density Functional Theory) or the like is relatively high in reliability and interpretation because the physical property such as energy of an electron system is calculated from a chemical background. On the other side, it takes a long calculation time, is difficult to apply to comprehensive material search, and is thus used for analysis for understanding the characteristic of the found material in the present circumstances. In contrast to this, a physical property prediction model development for a substance using a deep learning technique is rapidly developed in recent years.

However, as explained above, DFT takes a long calculation time. On the other hand, a model using the deep learning technique can predict the physical property value, but the existing model capable of inputting coordinates has difficulty in increasing the kinds of atoms and has difficulty in handling different states of a molecule, a crystal and so on and their coexisting state at the same time.

### SUMMARY

An Embodiment provides an inferring device and method improved in accuracy of inference of a physical property value of a substance system, and their training device and method.

According to an embodiment, the inferring device includes one or more memories and one or more processors. The one or more processors input a vector relating to an atom into a first network which extracts a feature of the atom in a latent space from the vector relating to the atom, and infer the feature of the atom in the latent space through the first network.

### BRIEF DESCRIPTION OF THE DRAWINGS

FIG. 1 is a schematic block diagram of an inferring device according to an embodiment;

FIG. 2 is a schematic diagram illustrating an atom feature acquirer according to an embodiment;

FIG. 3 is a view illustrating an example of coordinate setting of a molecule or the like according to an embodiment;

FIG. 4 is a view illustrating an example of acquiring graph data on a molecule or the like according to an embodiment;

FIG. 5 is a view illustrating an example of the graph data according to an embodiment;

FIG. 6 is a flowchart illustrating processing of the inferring device according to an embodiment;

FIG. 7 is a schematic block diagram of a training device according to an embodiment;

FIG. 8 is a schematic diagram of a composition in training of the atom feature acquirer according to an embodiment;

FIG. 9 is a chart illustrating examples of teacher data on physical property values according to an embodiment;

FIG. 10 is a chart illustrating an appearance of training the physical property values of the atom according to an embodiment;

FIG. 11 is a schematic block diagram of a structure feature extractor according to an embodiment;

FIG. 12 is a flowchart illustrating whole training processing according to an embodiment;

FIG. 13 is a flowchart illustrating processing of training of a first network according to an embodiment;

FIG. 14 is a chart illustrating an example of the physical property value by output from the first network according to an embodiment;

FIG. 15 is a flowchart illustrating processing of training of second, third, and fourth networks according to an embodiment;

FIG. 16 is a chart illustrating examples of output of the physical property values according to an embodiment; and

FIG. 17 is an implementation example of the inferring device and the training device according to an embodiment.

### DETAILED DESCRIPTION

Hereinafter, embodiments of the present invention will be explained referring to the drawings. The explanation of the drawings and embodiments is made as an example and does not limit the present invention.

[Inferring Device]

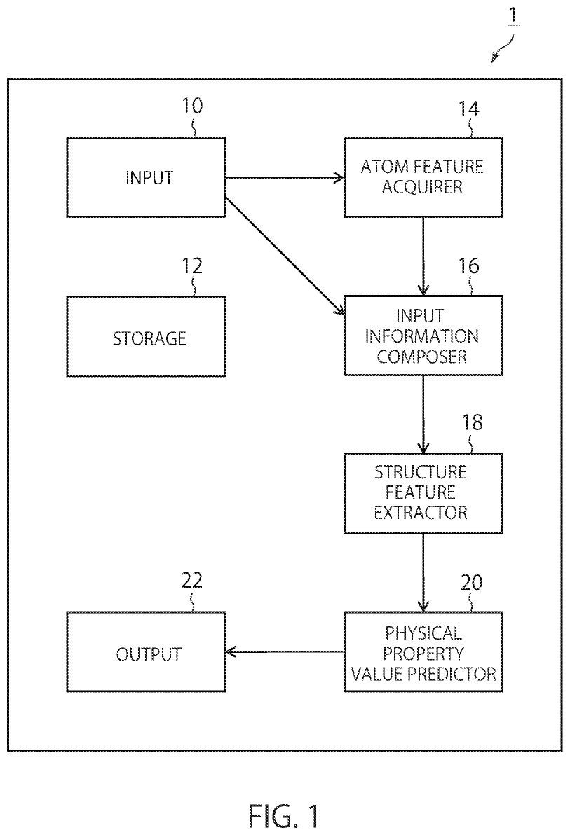

FIG. 1 is a block diagram illustrating the function of an inferring device 1 according to this embodiment. The inferring device 1 of this embodiment infers and outputs a physical property value of an inferring object which is a molecule or the like (hereinafter, the one including a monatomic molecule, a molecule, or a crystal is described as a molecule or the like) from information on the kind and information on coordinates of an atom, and information on a boundary condition. The inferring device 1 includes an input 10, a storage 12, an atom feature acquirer 14, an input information composer 16, a structure feature extractor 18, a physical property value predictor 20, and an output 22.

The inferring device 1 receives input of necessary information such as the kind and the coordinates of the atom, the boundary condition, and so on which are information on an inferring object being the molecule or the like via the input 10. In this embodiment, for example, the inferring device 1 is explained as the one which receives input of the information on the kind and the coordinates of the atom, and the boundary condition, and the information only needs to be, but not limited to, information which defines the structure of a substance whose physical property value is desired to be inferred.

The coordinates of the atom are three-dimensional coordinates of the atom, for example, in an absolute space of the like. For example, the coordinates may be coordinates in a coordinate system using a translation invariant and rotation invariant coordinate system. The coordinates are not limited to the above but only need to be coordinates using a coordinate system which can appropriately express the structure of the atoms in a substance such as the molecule or the like being the inferring object. Input of the coordinates of the atom can define at what relative position the atom exists in the molecule or the like.

As for the boundary condition, for example, in the case of desiring to acquire the physical property value of the inferring object being a crystal, input is the coordinates of an atom in a unit cell or in a supercell in which unit cells are repeatedly arranged. In this case, a case where when the input atom becomes a boundary surface with a vacuum, the same atomic arrangement is repeated neighboring thereto, is set. For example, such a boundary condition may be supposed that when a molecule is made closer to a crystal being a catalyst, a crystal face coming into contact with the molecule is a boundary with a vacuum and a crystal structure continues other than that. The inferring device 1 can infer not only the physical property value relating to the molecule but also the physical property relating to the crystal, the physical property value relating to both the crystal and the molecule, and so on.

The storage 12 stores information required for inference. For example, data used for inference input via the input 10 may be temporarily stored in the storage 12. Further, parameters required in respective modules, for example, parameters required for forming a neural network provided in respective modules and the like may be stored. Further, when information processing by software in the inferring device 1 is concretely realized using hardware resources, a program, an executable file and so on which are required for the software may be stored.

The atom feature acquirer 14 generates an amount indicating the feature of an atom. The amount indicating the feature of the atom may be expressed, for example, in a one-dimensional vector form. The atom feature acquirer 14 includes, for example, a neural network (first network) such as an MLP (Multilayer Perceptron) which, for example, when receiving input of a one-hot vector indicating an atom, transforms it into a vector in a latent space, and outputs the vector in the latent space as the feature of the atom.

Besides, the atom feature acquirer 14 may be the one which receives input of not the one-hot vector but other information such as a tensor, a vector or the like which indicates the atom. The one-hot vector, or the other information such as the tensor, the vector or the like is, for example, a symbol representing a focused atom or information similar to that. In this case, an input layer of the neural network may be formed as a layer having a dimension different from that using the one-hot vector.

The atom feature acquirer 14 may generate the feature for each inference, or may store an inferred result in the storage 12 as another example. For example, the feature may be stored in the storage 12 for a hydrogen atom, a carbon atom, an oxygen atom or the like which are frequently used, and the feature may be generated for each inference for the other atoms.

The input information composer 16, when receiving input of the input atom coordinates, the boundary condition, and the feature of the atom or the feature for discriminating the atom similar to the feature of the atom which are generated by the atom feature acquirer 14, transforms the structure of the molecule or the like into the format of a graph to adapt the transformed structure to the input to the network which processes a graph provided in the structure feature extractor 18.

The structure feature extractor 18 extracts the feature regarding the structure from the information on the graph generated by the input information composer 16. The structure feature extractor 18 includes a neural network on a graph basis such as a GNN (Graph Neural Network), GCN (Graph Convolutional Network) or the like.

The physical property value predictor 20 predicts a physical property value from the feature of the structure of the inferring object such as the molecule or the like extracted by the structure feature extractor 18 and outputs the physical property value. The physical property value predictor 20 includes, for example, a neural network such as MLP or the like. The characteristic or the like of the provided neural network sometimes differs depending on the physical property value desired to be acquired. Therefore, a plurality of different neural networks may be prepared in advance, and one of them may be selected according to the physical property value desired to be acquired.

The output 22 outputs the inferred physical property value. The output here is a concept including both of outputting it to the outside of the inferring device 1 via an interface and outputting it to the inside of the inferring device 1 such as the storage 12 or the like.

Each composition will be explained in more detail.

(Atom Feature Acquirer 14)

The atom feature acquirer 14 includes the neural network which, for example, when receiving input of the one-hot vector indicating an atom, outputs the vector in the latent space as explained above. The one-hot vector indicating an atom is, for example, is a one-hot vector indicating information on nuclear information. More specifically, the one-hot vector indicating an atom is, for example, the one made by transforming the proton number, the neutron number, and the electron number into a one-hot vector. For example, by inputting the proton number and the neutron number, an isotope can also be made an object whose feature is acquired. For example, by inputting the proton number and the electron number, an ion can also be made an object whose feature is acquired.

The data to be input may include information other than the above. For example, information such as an atomic number, a group in a periodic table, a period, a block, a half-life between isotopes may be added to the above one-hot vector and regarded as the input. Besides, the one-hot vector and another input may be combined as a one-hot vector in the atom feature acquirer 14. For example, a discrete value may be stored in the one-hot vector, and an amount (scalar, vector, tensor or the like) expressed by a continuous value may be added as the above input.

The one-hot vector may be separately generated by a user. As another example, an atomic name, an atomic number, another ID indicating an atom or the like is received as the input, and the atom feature acquirer 14 may separately include a one-hot vector generator which generates a one-hot vector referring to a database or the like from these kinds of information. Note that when the continuous value is added as the input, an input vector generator may be further provided which generates a vector other than the one-hot vector.

The neural network (first network) provided in the atom feature acquirer 14 may be, for example, an encoder portion of a model trained by the neural network forming an encoder and a decoder. The encoder and the decoder may be composed of a Variational Encoder Decoder which provides variance to the output from the encoder similarly to, for example, a VAE (Variational Autoencoder). An example of the case using the Variational Encoder Decoder will be explained below, and the encoder and the decoder are not limited to Variational Encoder Decoder, and only need to be a model such as a neural network which can appropriately acquire the vector in the latent space for the feature of an atom, namely, the feature amount.

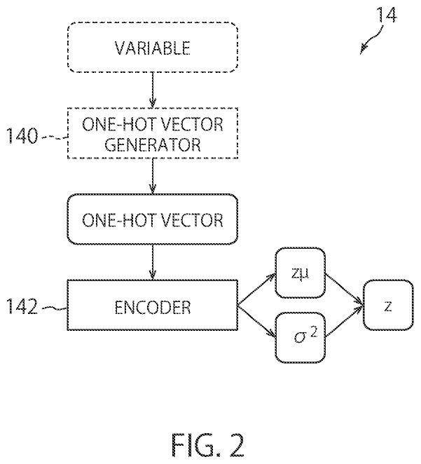

FIG. 2 is a diagram illustrating the concept of the atom feature acquirer 14. The atom feature acquirer 14 includes, for example, a one-hot vector generator 140 and an encoder 142. The encoder 142 and a later-explained decoder are a partial composition of the network by the above Variational Encoder Decoder. Note that the encoder 142 is illustrated, and another network, an arithmetic unit and the like for outputting the feature amount may be inserted after the encoder 142.

The one-hot vector generator 140 generates a one-hot vector from a variable indicating an atom. The one-hot vector generator 140 when, for example, receiving input of a value to be transformed to the one-hot vector such as the proton number or the like, generates the one-hot vector using the input data.

When the input data is an indirect value such as the atomic number, the atomic name, or the like, the one-hot vector generator 140 acquires the value of the proton number or the like, for example, from the database or the like inside or outside the inferring device 1, and generate the one-hot vector. The one-hot vector generator 140 performs appropriate processing based on the input data as above.

As explained above, the one-hot vector generator 140, when directly receiving input of the input information to be transformed to the one-hot vector, transforms each of variables into the format compatible with the one-hot vector and generates the one-hot vector On the other hand, the one-hot vector generator 140 may automatically acquire, in the case where only the atomic number is input, data required for the transformation of the one-hot vector from the input data, and may generate the one-hot vector based on the acquired data.

Note that though the use of the one-hot vector in the input is described in the above, this is described as an example, and this embodiment is not limited to this aspect. For example, a vector, a matrix, a tensor or the like not using the one-hot vector can also be used as the input.

Note that when the one-hot vector is stored in the storage 12, the one-hot vector may be acquired from the storage 12, or when the user separately prepares the one-hot vector and inputs it into the inferring device 1, the one-hot vector generator 140 is not an essential composition.

The one-hot vector is input into the encoder 142. The encoder 142 outputs a vector z_{μ} indicating an average value of the vectors being the features of atoms and a vector σ² indicating the variance of the vectors z_{μ}, from the input one-hot vector. The one sampled from the output result is a vector z. For example, in training, the feature of the atom is recomposed from the vector z_{μ}.

The atom feature acquirer 14 outputs the generated vector z_{μ} to the input information composer 16. Note that it is possible to use Reparametrization trick which is used as one method of VAE, and in this case, the vector z may be found as follows using a vector ε being a random value. Note that a mark odot (dot in a circle) indicates an element-wise product of the vector.

[Math 1]

z=z_{μ}+σ²⊙∈ (1)

As another example, z having no variance may be output as the feature of the atom.

As will be explained later, the first network is trained, when receiving input of the one-hot vector or the like of the atom, as a network including an encoder which extracts the feature and a decoder which outputs the physical property value from the feature. The use of the appropriately trained atom feature acquirer 14 makes it possible for the network to extract the information required for the prediction of the physical property value of the molecule or the like without the user selecting the information.

The use of the encoder and the decoder can advantageously utilize more information in that the information can be used even if the physical property values required for all atoms are unknown, as compared with the case of directly inputting the physical property value. Further, because of mapping in the continuous latent space, atoms closer in property are transferred near to each other and atoms different in property are transferred far from each other in the latent space, so that an atom can be interpolated between them. Therefore, even if all of the atoms are not included in learning data, it is possible to output a result by the interpolation between atoms. Even if the learning data for some of atoms is not sufficient, it is possible to generate the feature capable of outputting the physical property value with high accuracy.

As explained above, the atom feature acquirer 14 is composed including, for example, the neural network (first network) capable of extracting the feature capable of decoding the physical property value of each atom. Via the encode of the first network, it is also possible to transform, for example, the one-hot vector of the dimensions of 10² or more order to the feature amount vector of about 16 dimensions. The first network is composed including the neural network having an output dimension lower than an input dimension as explained above.

(Input Information Composer 16)

The input information composer 16 generates a graph relating to the atomic arrangement and the connection in the molecule or the like based on the input data and the data generated by the atom feature acquirer 14. The input information composer 16 determines the presence or absence of an neighboring atom in consideration of the boundary condition together with the structure of the molecule or the like to be input, and decides the coordinates of the neighboring atom if it exists.

For example, in the case of a monomolecule, the input information composer 16 generates a graph utilizing the atom coordinates indicated in the input as the neighboring atom. In the case of a crystal, the input information composer 16 decides, for example the coordinates from the input atom coordinates for the atom in the unit cell, and decides the coordinates of an outside neighboring atom from the repeated pattern of the unit cell for the atom located at an outer rim of the unit cell. In the case where an interface exists in the crystal, for example, the neighboring atom is decided without applying the repeated pattern for the interface side.

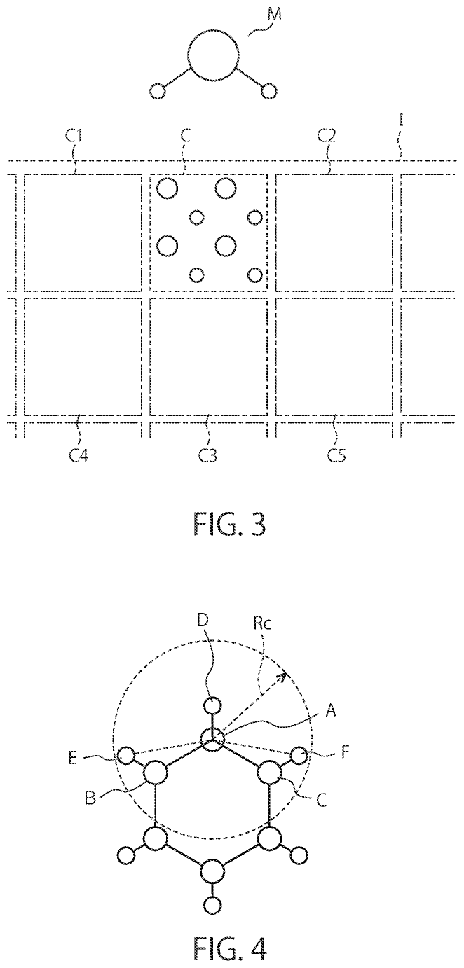

FIG. 3 is a view illustrating an example of coordinate setting according to this embodiment. For example, in the case of generating a graph of only a molecule M, a graph is generated from kinds of three atoms constituting the molecule M and their relative coordinates.

For example, in the case of generating a graph of only a crystal having a repetition and having an interface I, the graph is created while assuming a repetition C1 of a unit cell C of the crystal to the right side, a repetition C2 to the left side, a repetition C3 to the lower side, a repetition C4 to the lower left side, a repetition C5 to the lower right side, . . . and assuming neighboring atoms to the respective atoms. In the drawing, a dotted line indicates the interface I, a unit cell indicated by a broken line indicates the input structure of the crystal, and a region indicated by a one-dotted chain line indicates a region assuming the repetition of the unit cell C of the crystal. In short, the graph is created while assuming the neighboring atoms to the respective atoms constituting the crystal in a range not exceeding the interface I.

In the case of desiring to infer the physical property value when the molecule acts on the crystal as in a catalyst or the like, the graph is created by assuming the repetition in consideration of the molecule M and the interface I of the above crystal and calculating the coordinates of the neighboring atom from each of the atoms constituting the molecule and the neighboring atom from each of the atoms constituting the crystal.

Note that since there is a limit in size of the graph to be input, for example, the interface I, the unit cell C, and the repetition of the unit cell C may be set so that the molecule M comes to be located at the center. In other words, the graph may be created by appropriately executing the repetition of the unit cell C and acquiring the coordinates. For creating the graph, for example, the repetition to up, down, left, and right of the unit cell C so as not to exceed the atomicity which can be expressed by the graph in a range not exceeding the interface with the unit cell C closest to the molecule M as a center is assumed to acquire the coordinates of the respective neighboring atoms.

One unit cell C of the crystal having the interface I is input for one molecule M in FIG. 3, but not limited to this. For example, there may be a plurality of molecules M or there may be a plurality of crystals.

Further, the input information composer 16 may calculate a distance between two atoms composed in the above and an angle formed when a certain atom of three atoms is regarded as a vertex. The distance and the angle are calculated based on the relative coordinates of the atoms. The angle is acquired using, for example, an inner product and the cosine theorem of vectors. For example, they may be calculated for a combination of all atoms, or the input information composer 16 may decide a cutoff radius Rc, search for other atoms existing in the cutoff radius Rc for each atom, and calculate a combination of atoms existing in the cutoff radius Rc.

An index may be given to each of the composing atoms, and the calculated results of them may be stored in the storage 12 together with the combination of the indexes. When calculating, the structure feature extractor 18 may read those values from the storage 12 at the timing when using the values, or the input information composer 16 may output those values to the structure feature extractor 18.

Besides, the molecule or the like is two-dimensionally illustrated for understanding, but exists in a three-dimensional space as a matter of course. Therefore, the repetition condition is also applied to the front side and the back side of the drawing in some cases.

The input information composer 16 creates a graph being the input to the neural network from the information on the input molecule or the like and the feature of each atom generated by the atom feature acquirer 14 as above.

(Structure Feature Extractor 18)

The structure feature extractor 18 in this embodiment includes a neural network which, when receiving input of the graph information, outputs the feature regarding the structure of the graph as explained above. Here, the feature of the graph to be input may include angular information.

The structure feature extractor 18 is designed to keep an invariant output, for example, with respect to the replacement of the same kind of atom in the input graph, the translation and rotation of the input structure. These are caused from the fact that the physical property of an actual substance does not depend on these amounts. For example, the definition of the neighboring atoms and the angle among three atoms as below enables input of the information on the graph to satisfy these conditions.

First, for example, the structure feature extractor 18 decides a maximum neighboring atomicity Nn and the cutoff radius Rc, and acquires the neighboring atoms to an atom A on which attention is focused (focused atom). By setting the cutoff radius Rc, it is possible to exclude atoms whose influences exerted on each other are small enough to be negligible, and to prevent the number of atoms extracted as the neighboring atoms from being too many. Further, by performing graph convolution a plurality of times, it becomes possible to capture the influence of atoms exiting outside the cutoff radius.

When the neighboring atomicity is less than the maximum neighboring atomicity Nn, atoms of the same kind as the atom A are randomly arranged as dummies at positions sufficiently far from the cutoff radius Rc. When the neighboring atomicity is more than the maximum neighboring atomicity Nn, Nn atoms are selected, for example, in an order of being closer in distance to the atom A are selected and made candidates for the neighboring atoms. In consideration of the neighboring atoms, the combinations of three atoms are _{Nn}C₂ combinations. For example, when Nn=12, ₁₂C₂=66 combinations.

The cutoff radius Rc relates to an interaction distance of a physical phenomenon to be reproduced. In the case of a close-packed system such as a crystal, when using 4 to 8×10⁻⁸ cm as the cutoff radius Rc, sufficient accuracy can be secured in many cases. On the other hand, in the case of considering the interaction between a crystal surface and a molecule, between molecules or the like, these two are not connected in terms of structure, and therefore the influence of a far atom cannot be taken into consideration even if the graph convolution is repeated, so that the cutoff radius becomes a direct maximum interaction distance. Also in this case, 8×10⁻⁸ cm or more is considered as the cutoff radius Rc and the initial shape is started from the distance, whereby the cutoff radius Rc can be applied.

As the maximum neighboring atomicity Nn, about 12 is selected from the viewpoint of calculation efficiency but not limited to this. For the atoms within the cutoff radius Rc which are not selected as the Nn neighboring atoms, their influences can be considered by repeating the graph convolution.

For one focused atom, for example, the feature of the atom, the features of two neighboring atoms, the distances between the atom and the two atoms, and the value of the angle formed between the two neighboring atoms with the atom as a center are concatenated to be regarded as one set of input. The feature of the atom is regarded as the feature of a node, and the distances and the angle are regarded as the feature of an edge. For the feature of the edge, the acquired numerical value can be used as it is, but may be subjected to predetermined processing. For example, the numerical value may be subjected to binning into a specific width or further subjected to the Gaussian filter.

FIG. 4 is a view for explaining an example of how to acquire data on a graph. The focused atom is considered as the atom A. The atoms are two-dimensionally illustrated as in FIG. 3 but, more specifically, the atoms exist in the three-dimensional space. In the following explanation, it is supposed that the candidates for the neighboring atoms to the atom A are atoms B, C, D, E, F, but the number of atoms is not limited to this because the number of atoms is decided by Nn and the candidates for the neighboring atoms change depending on the structure of the molecule or the like and the existing state. For example, when more atoms G, H, . . . and so on exit, the following feature extraction and so on are similarly executed in a range without exceeding Nn.

An arrow with a dotted line from the atom A indicates the cutoff radius Rc. A range indicated by the cutoff radius Rc from the atom A is a range of a circle indicated by a dotted line. The neighboring atoms to the atom A are searched for in the circle of the dotted line. When the maximum neighboring atomicity Nn is 5 or more, the five atoms B, C, D, E, F are determined as the neighboring atoms to the atom A. As in the above manner, the data on the edge is generated for atoms which are connected in the structural formula and also for atoms which are not connected in the structural formula in the range formed by the cutoff radius Rc.

The structure feature extractor 18 extracts a combination of atoms for acquiring the angular data with the atom A as a vertex. Hereinafter, the combination of the atoms A, B, C is described as A-B-C. The combinations to the atom A are ₅C₂=10 combinations such as A-B-C, A-B-D, A-B-E, A-B-F, A-C-D, A-C-E, A-C-F, A-D-E, A-D-F, A-E-F. The structure feature extractor 18 may give, for example, an index to each of them. The index may be the one focusing on only the atom A, or may be uniquely given in consideration of the one focusing on a plurality of atoms or all of atoms. By giving the index in this manner, it becomes possible to uniquely designate the combination of the focused atom and the neighboring atoms.

It is assumed that the index of the combination of A-B-C is, for example, 0. The graph data in which the combination of neighboring atoms is the atom B and the atom C, namely, the graph data of an index 0 is generated for each of the atom B and the atom C.

It is assumed that, for example, the atom B is a first neighboring atom and the atom C is a second neighboring atom to the atom A being the focused atom. As the data relating to the first neighboring atom, the structure feature extractor 18 concatenates the information on the feature of the atom A, the feature of the atom B, the distance between the atoms A and B, and the angle formed among the atoms B, A, C. As the data relating to the second neighboring atom, the structure feature extractor 18 concatenates the information on the feature of the atom A, the feature of the atom C, the distance between the atoms A and B, and the angle formed among the atoms C, A, B.

As the distance between the atoms and the angle formed among the three atoms, those calculated by the input information composer 16 may be used, or when the input information composer 16 does not calculate them, the structure feature extractor 18 may calculate them. For the calculation of the distance and the angle, the method similar to that explained for the input information composer 16 can be used. Further, the timing of the calculation may be dynamically changed such that when the atomicity is larger than a predetermined number, the structure feature extractor 18 calculates them, or when the atomicity is smaller than the predetermined number, the input information composer 16 calculates them. In this case, which of the structure feature extractor 18 and the input information composer 16 calculates them may be decided based on the state of the resource such as a memory, a processor or the like. Hereinafter, the feature of the atom A when focusing on the atom A is described as a node feature of the atom A. In the above case, the data on the node feature of the atom A is redundant, and therefore may be collectively held. For example, the graph data on the index 0 may be composed including information on the node feature of the atom A, the feature of the atom B, the distance between the atoms A and B, the angle among the atoms B, A, C, the feature of the atom C, the distance between the atoms A and C, and the angle among the atoms C, A, B.

The distance between the atoms A and B, the angle among the atoms B, A, C are collectively described as an edge feature of the atom B, and the distance between the atoms A and C and the angle among the atoms C, A, B are similarly collectively described as an edge feature of the atom C. The edge feature includes the angular information and is thus an amount different depending on the atom being the mate of the combination. For example, the edge feature of the atom B when the neighboring atoms are B, C to the atom A and the edge feature of the atom B when the neighboring atoms are B, D have different values.

The structure feature extractor 18 generates the data on all of the combinations of two atoms being the neighboring atoms similarly to the above-explained graph data on the atom A, for all of the atoms.

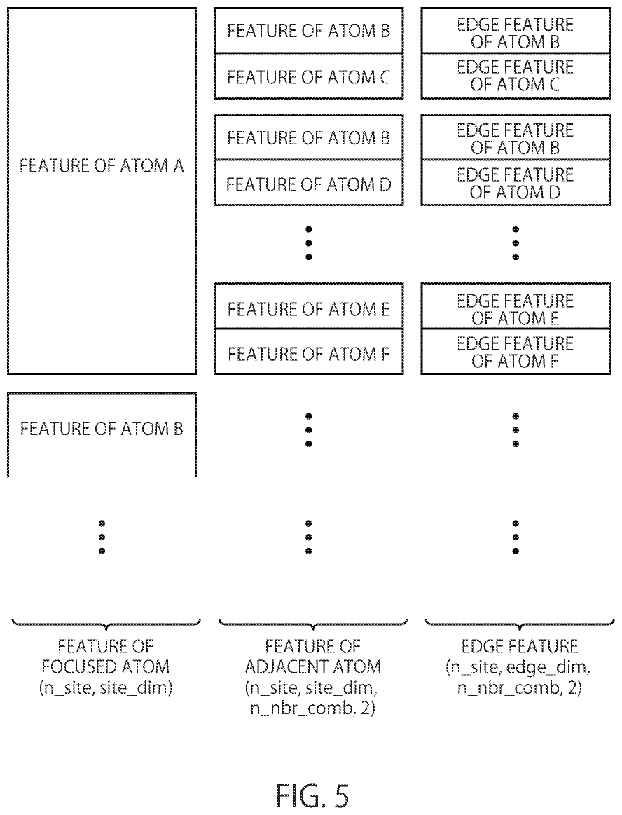

FIG. 5 illustrates an example of the graph data generated by the structure feature extractor 18.

For the node feature of the atom A being the first atom or the focused atom, the features and the edge features of the atoms are generated for the combinations of the neighboring atoms existing in the cutoff radius Rc from the atom A. The horizontal connection in the drawing may be linked, for example, by an index. Similarly to that the neighboring atoms to the atom A being the first focused atom are selected to acquire the features, the features are acquired for the combinations of the second, third and more neighboring atoms also for the atoms B, C, . . . as the second, third and more focused atoms.

As above, the node features, and the features and the edge features of atoms relating to the neighboring atoms are acquired for all of the atoms. As a result of this, the feature of the focused atom is a tensor of (n_site, site_dim), the feature of the neighboring atom is a tensor of (n_site, site_dim, n_nbr_comb, 2), and the edge feature is a tensor of (n_site, edge_dim, n_nbr_comb, 2). Note that n_site is the atomicity, site_dim is the dimension of the vector indicating the feature of the atom, n_nbr_comb is the number of combinations (=_{Nn}C₂) of the neighboring atoms to the focused atom, and edge_dim is the dimension of the edge feature. The feature of the neighboring atom and the edge feature are acquired for each of the neighboring atoms by selecting the two neighboring atoms to the focused atom, and therefore become tensors having dimensions of twice (n_site, site_dim, n_nbr_comb) and (n_site, edge_dim, n_nbr_comb), respectively.

The structure feature extractor 18 includes a neural network which, when receiving input of these kinds of data, outputs the feature of the atom and the edge feature after updating them. In other words, the structure feature extractor 18 includes a graph data acquirer which acquires data on a graph, and a neural network which, when receiving input of the data relating to the graph, updates the data relating to the graph. The neural network includes a second network which outputs the node feature of (n_site, site_dim) dimensions and a third network which outputs the edge feature of (n_site, edge_dim, n_nbr_comb, 2) dimensions, from the data having (n_site, site_dim+edge_dim+site_dim, n_nbr_comb, 2) dimensions being the input data.

The second network includes a network which, when receiving input of the tensor including the feature of the neighboring atom for two atoms to the focused atom, reduces it in dimension to a tensor of (n_site, site_dim, n_nbr_comb, 1) dimensions, and a network which, when receiving input of the tensor including the feature of the neighboring atom reduced in dimension with respect to the focused atom, reduces it in dimension to a tensor of (n_site, site_dim, 1, 1) dimensions.

A first-stage network of the second network transforms the feature to each of the neighboring atoms when the atoms B, C with respect to the atom A being the focused atom are regarded as the neighboring atoms, to the feature about the combination of the neighboring atoms B, C with respect to the atom A being the focused atom. This network enables extraction of the feature of the combination of the neighboring atoms. The network transforms the combinations of all of the neighboring atoms with respect to the atom A being the first focused atom to this feature. Further, the network similarly transforms the combinations of all of the neighboring atoms with respect to the atom B, . . . being the second focused atom. This network transforms the tensor indicating the feature of the neighboring atom from the (n_site, site_dim, n_nbr_comb, 2) dimensions to the (n_site, site_dim, n_nbr_comb, 1) dimensions.

A second-stage network of the second network extracts the node feature of the atom A having the features of the neighboring atoms from the combination of the atoms B, C, the combination of the atoms B, D, . . . , the combination of the atoms E, F with respect to the atom A. This network enables extraction of the node features in consideration of the combination of the neighboring atoms with respect to the focused atom. Further, the network similarly extracts the node features in consideration of all of the combinations of the neighboring atoms for the atom B. This network transforms the output from the second-stage network from the (n_site, site_dim, n_nbr_comb, 1) dimensions to the (n_site, site_dim, 1, 1) dimensions which are the dimensions equivalent to the dimensions of the node feature.

The structure feature extractor 18 in this embodiment updates the node feature based on the output from the second network. For example, the structure feature extractor 18 adds the output from the second network and the node feature to acquire the node feature which has been updated (hereinafter, described as an updated node feature) via an activation function such as tan h( ). Besides, this processing does not need to be provided separately from the second network in the structure feature extractor 18, and the addition and the activation function processing may be provided as a layer on an output side of the second network. Further, the second network can reduce the information which can be unnecessary to the finally acquired physical property value as in a later-explained third network.

The third network is a network which, when receiving input of the edge feature, outputs an edge feature which has been updated (hereinafter, described as an updated edge feature). The third network transforms the tensor of the (n_site, edge_dim, n_nbr_comb, 2) dimensions to the tensor of the (n_site, edge_dim, n_nbr_comb, 2) dimensions. For example, the third network reduces the information which is unnecessary to the finally acquired physical property value desired to be finally acquired by using a gate or the like. The third network having this function is generated by training parameters by a later-explained training device. The third network may include a network having the same input and output dimensions as a second stage in addition to the above.

The structure feature extractor 18 in this embodiment updates the edge feature based on the output from the third network. The structure feature extractor 18 adds, for example, the output from the third network and the edge feature to acquire the updated edge feature via the activation function such as the tan h( ). Further, when a plurality of features to the same edge are extracted, an average value of them may be calculated and made into one edge feature. These kinds of processing do not need to be provided separately from the third network in the structure feature extractor 18, and the addition and the activation function processing may be provided as a layer on the output side of the third network.

Each of the networks of the second network and the third network may be formed by a neural network appropriately using, for example, a convolution layer, batch normalization, pooling, gate processing, activation function and so on. Not limited to the above, each of the networks may be formed by MLP or the like. Besides, each of the networks may be a network having an input layer into which a tensor made by squaring each element of the input tensor can further be input.

Further, as another example, the second network and the third network are not networks formed separately but may be formed as one network. In this case, the networks are formed as a network which, when receiving input of the node feature, the feature of the neighboring atom, and the edge feature, outputs the updated node feature and edge feature according to the above example.

The structure feature extractor 18 generates the data relating to the node and the edge of the graph in consideration of the neighboring atom based on the input information composed by the input information composer 16, and updates the generated data to update the node feature and the edge feature of each atom. The updated node feature is a node feature in consideration of the neighboring atom. The updated edge feature is an edge feature made by deleting the information which can be extra information relating to the physical property value desired to be acquired from the generated edge feature.

(Physical Property Value Predictor 20)

The physical property value predictor 20 in this embodiment includes a neural network (fourth network) such as MLP which, when receiving input of the feature relating to the structure of the molecule or the like, for example, the updated node feature and the updated edge feature, predicts and outputs the physical property value as explained above. The updated node feature and the updated edge feature are not only input as they are but may also be input after being processed according to the physical property value desired to be obtained as will be explained later.

The network used for the prediction of the physical property value may be changed, for example, by the nature of the physical property desired to be predicted. For example, when energy is desired to be acquired, the feature for each node is input into the same fourth network, the acquired output is regarded as the energy of each atom, and the total value of energies is output as a total energy value.

In the case of predicting the characteristic between predetermined atoms, the updated edge feature is input into the fourth network to predict the physical property value desired to be acquired.

In the case of predicting the physical property value decided from the whole input, the average, total or the like of the updated node features is calculated, and the calculated value is input into the fourth network to predict the physical property value.

As explained above, the fourth network may be composed as a network different with respect to the physical property value desired to be acquired. In this case, at least one of the second network and the third network may be formed as a neural network which extracts the feature amount to be used for acquiring the physical property value.

As another example, the fourth network may be formed as a neural network which outputs a plurality of physical property values at the same timing as its output. In this case, at least one of the second network and the third network may be formed as a neural network which extracts the feature amount to be used for acquiring a plurality of physical property values.

As explained above, the second network, the third network, and the fourth network may be formed as neural networks different in parameter, shape of a layer and the like depending on the physical property value desired to be acquired, and may be trained based on the physical property values.

The physical property value predictor 20 appropriately processes the output from the fourth network based on the physical property value desired to be acquired, and outputs the resultant. For example, in the case of finding the whole energy, when the energy of each of atoms is acquired by the fourth network, their energies are totaled and output. Also in the case of the other example, the value output from the fourth network is similarly subjected to appropriate processing for the physical property value desired to be acquired and used as the output value.

The amount output from the physical property value predictor 20 via the output 22 is output to the outside or the inside of the inferring device 1.

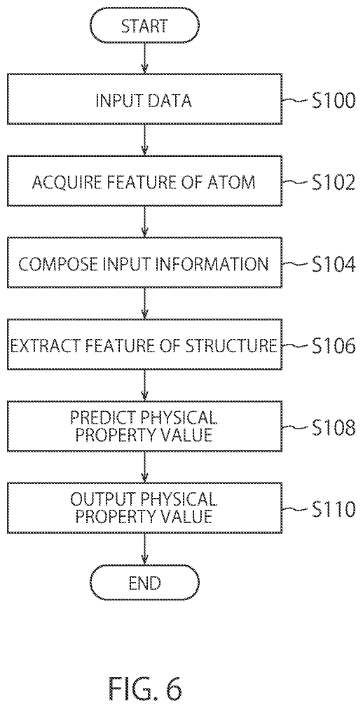

FIG. 6 is a flowchart illustrating the flow of processing of the inferring device 1 according to this embodiment. The entire processing of the inferring device 1 will be explained using the flowchart. Detailed explanation of each step is as described above.

First of all, the inferring device 1 of this embodiment accepts input of data via the input 10 (S100). The information to be input is the boundary condition of the molecule or the like, the structure information on the molecule or the like, and the information on the atoms constituting the molecule of the like. The boundary condition of the molecule or the like and the structure information on the molecule or the like may be designated by the relative coordinates of the atoms.

Next, the atom feature acquirer 14 generates the feature of each of the atoms constituting the molecule or the like from the input information on the atoms used for the molecule or the like (S102). As explained above, the features of various atoms may be generated in advance by the atom feature acquirer 14 and stored in the storage 12 or the like. In this case, the feature may be read from the storage 12 based on the kind of the atom to be used. The atom feature acquirer 14 inputs the information on the atom into the trained neural network included in itself and thereby acquires the feature of the atom.

Next, the input information composer 16 composes information for generating the graph information on the molecule or the like from the input boundary condition, coordinates, and features of the atoms (S104). For example, as in the example illustrated in FIG. 3, the input information composer 16 generates information describing the structure of the molecule or the like.

Next, the structure feature extractor 18 extracts the feature of the structure (S106). The extraction of the feature of the structure is executed by two kinds of processing such as generation processing of the node feature and the edge feature about each of the atoms of the molecule or the like and update processing of the node feature and the edge feature. The edge feature includes information on an angle formed between two neighboring atoms with the focused atom as a vertex. The generated node feature and edge feature are extracted as the updated node feature and the updated edge feature respectively through the trained neural network.

Next, the physical property value predictor 20 predicts the physical property value from the updated node feature and the updated edge feature (S108). The physical property value predictor 20 outputs information from the updated node feature and the updated edge feature through the trained neural network and predicts the physical property value based on the output information.

Next, the inferring device 1 outputs the inferred physical property value to the outside or the inside of the inferring device 1 via the output 22 (S110). As a result of this, it becomes possible to infer and output the physical property value based on information including the information on the feature of the atom in the latent space and the angular information between the neighboring atoms in consideration of the boundary condition in the molecule or the like.

As in the above, according to this embodiment, it becomes possible to infer the physical property value with high accuracy by extracting the updated node feature and edge feature including the features of the neighboring atoms using the graph data including the node feature including the feature of the atom and the edge feature including the angular information formed by two neighboring atoms, based on the boundary condition, the arrangement of atoms in the molecule or the like, and the extracted feature of the atom, and inferring the physical property value using the extraction result. Since the feature of the atom is extracted as in the above, the same inferring device 1 can be easily applied also in the case of increasing the kinds of atoms.

Note that in this embodiment, differentiable operations are combined to obtain the output. In other words, it is possible to go back to the information on each atom from the output inference result. For example, when a whole energy P in the input structure is inferred, the force acting on each atom can be calculated by calculating the differentiation of the input coordinates in the inferred whole energy P. This differentiation can be executed without any problem because the neural network is used and other operations are also executed by differentiable operations as will be explained later. By acquiring the force acting on each atom as above, it becomes possible to perform structure relaxation or the like using the force at high speed. Further, it becomes possible to replace DFT calculation by calculation of the energy, for example, using the coordinates as input and N-order automatic differentiation. Further, it is similarly possible to easily acquire the differential operation expressed by Hamiltonian or the like from the output of the inferring device 1 and to execute analysis of various physical properties at higher speed.

The use of the inferring device 1 enables execution of search for a material having a desired physical property value about various molecules or the like, more specifically, molecules or the like having various structures, molecules or the like including various atoms. For example, it is also possible to search for a catalyst of the like high in reactivity to a certain compound.

[Training Device]

A training device according to this embodiment trains the above-explained inferring device 1. The training device trains especially the neural networks provided in the atom feature acquirer 14, the structure feature extractor 18, and the physical property value predictor 20 of the inferring device 1, respectively.

Note that the training means generation of a model having a structure such as the neural network or the like and capable of appropriate output to the input in this description.

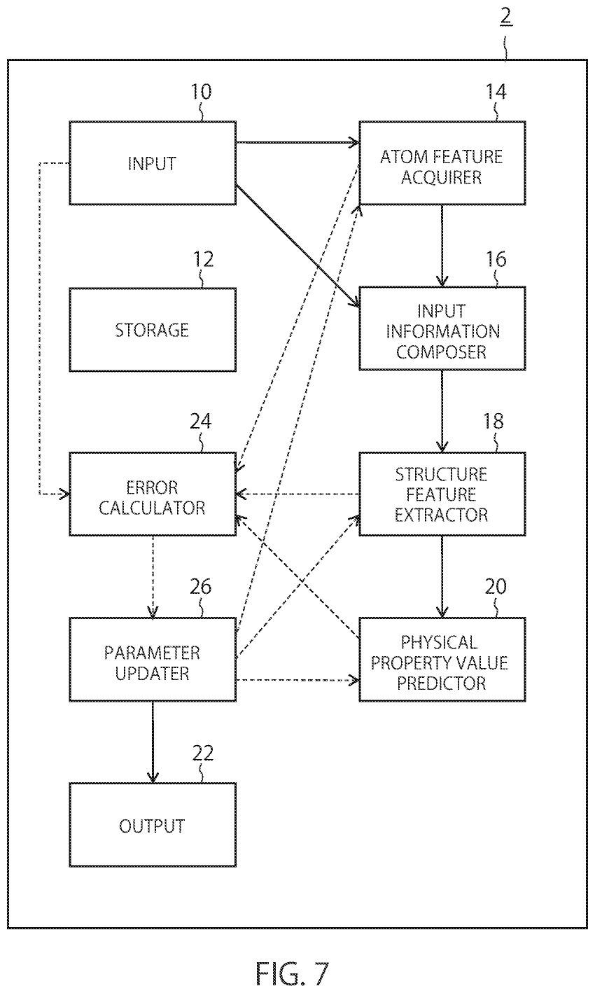

FIG. 7 is an example of a block diagram of a training device 2 according to this embodiment. The training device 2 includes an error calculator 24 and a parameter updater 26 in addition to the atom feature acquirer 14, the input information composer 16, the structure feature extractor 18, and the physical property value predictor 20 included in the inferring device 1. The input 10, the storage 12, and the output 22 may be common to the inferring device 1 or may be inherent to the training device 2. Detailed explanation of the same compositions as those of the inferring device 1 will be omitted.

The flow indicated by a solid line is processing of forward propagation, and the flow indicated by a broken line is processing of backward propagation.

The training device 2 receives input of training data via the input 10. The training data is output data which becomes input data and teacher data.

The error calculator 24 calculates an error between the teacher data in the atom feature acquirer 14, the structure feature extractor 18, and the physical property value predictor 20, and, the output from each neural network. The methods for calculating the error for the neural networks are not limited to the same operation, but may be appropriately selected based on the parameters being respective update objects or the network compositions.

The parameter updater 26 propagates backward the error in each neural network based on the error calculated by the error calculator 24 to update the parameter of the neural network. The parameter updater 26 may perform comparison with the teacher data through all of the neural networks or may update the parameter using the teacher data for each neural network.

Each of the above-explained modules of the inferring device 1 can be formed by a differentiable operation. Therefore, it is possible to calculate the gradient in the order of the physical property value predictor 20, the structure feature extractor 18, the input information composer 16, and the atom feature acquirer 14, and to appropriately propagate backward the error at a position other than the neural network.

For example, in the case where the whole energy is desired to be inferred as the physical property value, it is possible to express the whole energy P=τᵢ Fᵢ(xᵢ, yᵢ, zᵢ, Aᵢ) using (xᵢ, yᵢ, zᵢ) as the coordinates (relative coordinates) of an i-th atom and A as the feature of the atom. In this case, the differential value of dP/dxᵢ or the like can be defined for all of the atoms, thus enabling the error backward propagation from the output to the calculation of the feature of the atom in the input.

Besides, as another example, the modules may be individually optimized. For example, the first network included in the atom feature acquirer 14 can be generated by optimizing the neural network capable of extracting the physical property value from the one-hot vector using an identifier of the atom and the physical property value. Hereinafter, the optimization of the networks will be explained.

(Atom Feature Acquirer 14)

The first network of the atom feature acquirer 14 can be trained to output a characteristic value, for example, when receiving input of the identifier of the atom or the one-hot vector. The neural network may be the one which uses, for example, Variational Encoder Decoder based on VAE as explained above.

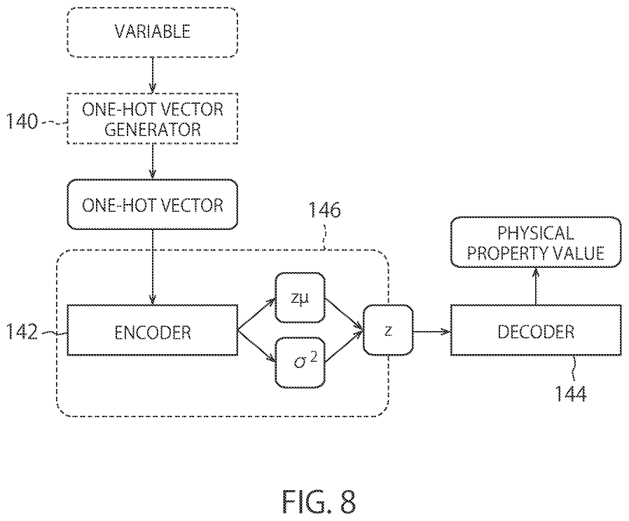

FIG. 8 is a formation example of the network used in the training of the first network. For example, a first network 146 may use the encoder 142 portion of Variational Encoder Decoder including the encoder 142 and a decoder 144.

The encoder 142 is a neural network which outputs the feature in the latent space for each kind of atom, and is the first network used in the inferring device 1.

The decoder 144 is a neural network which outputs the physical property value when receiving input of the vector in the latent space output from the encoder 142. By connecting the decoder 144 behind the encoder 142 and performing supervised learning, it becomes possible to execute the training of the encoder 142.

Into the first network 146, the one-hot vector expressing the property of the atom is input as explained above. The one-hot vector generator 140 may be provided which, when receiving input of the atomic number, the atomic name, or the like or the value indicating the property of each atom, generates the one-hot vector as in the above.

The data to be used as the teacher data is, for example, various physical property values. The physical property values may be acquired, for example, from the chronological scientific tables or the like.

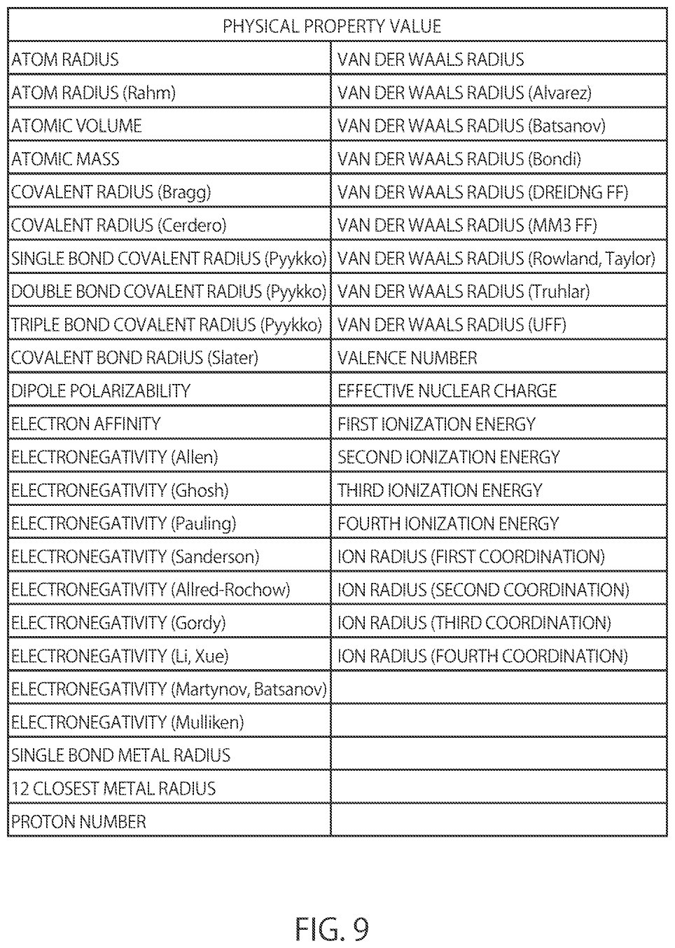

FIG. 9 is a table indicating examples of the physical property values. For example, the properties of the atom listed in this table are used as the teacher data for output from the decoder 144.

The ones with parentheses in the table are found by the methods described in the parentheses. Further, as the ion radii, the first coordination to the fourth coordination are used. As concrete examples, the ion radii at second, third, fourth, sixth coordinations are listed in order for oxygen.

The neural network including the encoder 142 and the decoder 144 illustrated in FIG. 8, when receiving input of the one-hot vector indicating an atom, performs optimization to output, for example, the properties listed in FIG. 9. This optimization is performed by the error calculator 24 calculating the loss between the output value and the teacher data and the parameter updater 26 executing backward propagation based on the loss to find the gradient and update the parameter. By performing the optimization, the encoder 142 functions as a network which outputs the vector in the latent space from the one-hot vector, and the decoder 144 functions as a network which outputs the physical property value from the vector in the latent space.

For the update of the parameter, for example, Variational Encoder Decoder is used. As explained above, a method of Reparametrization trick may be used.

After the end of the optimization, the neural network forming the encoder 142 is regarded as the first network 146, and the parameter for the encoder 142 is acquired. The value to be output may be a vector of z_{μ} illustrated in FIG. 8 or may be a value in consideration of a variance σ². Besides, as another example, both of z_{μ} and σ² may be output so that both of z_{μ} and σ² are input into the structure feature extractor 18 of the inferring device 1. In the case of using a random number, for example, a fixed random number table may be used so as to make processing which can be propagated backward.

Note that the physical property values of the atom listed in the table in FIG. 9 are examples and all of the physical property values do not need to be used, and physical property values other than those listed in this table may be used.

In the case of using various physical property values, predetermined physical property values do not exist in some cases depending on the kind of atom. For example, in the case of a hydrogen atom, the second ionization energy does not exist. In this case, for example, the optimization of the network may be executed on the condition that this value does not exist. Even when there is a non-existing value, it is possible to generate the neural network which outputs the physical property values. Even when all of the physical property values cannot be input as in the above, the atom feature acquirer 14 according to this embodiment can generate the feature of the atom.

Further, by generating the first network 146 as above, the one-hot vector is mapped in the continuous space, so that the atoms close in property are transferred to be close to each other in the latent space and the atoms remarkably different in property are transferred to be far from each other in the latent space. Therefore, for the atoms between them, results can be output by interpolation even if their properties do not exist in the teacher data. Further, even when the learning data is not sufficient for some of atoms, their features can be inferred.

It is also possible to input the atom feature vector extracted as above into the inferring device 1. Even if the learning data amount is not sufficient or lacks for some of atoms in training of the inferring device 1, the inference can be executed by interpolation of the interatomic feature. Further, the data amount required for training can also be reduced.

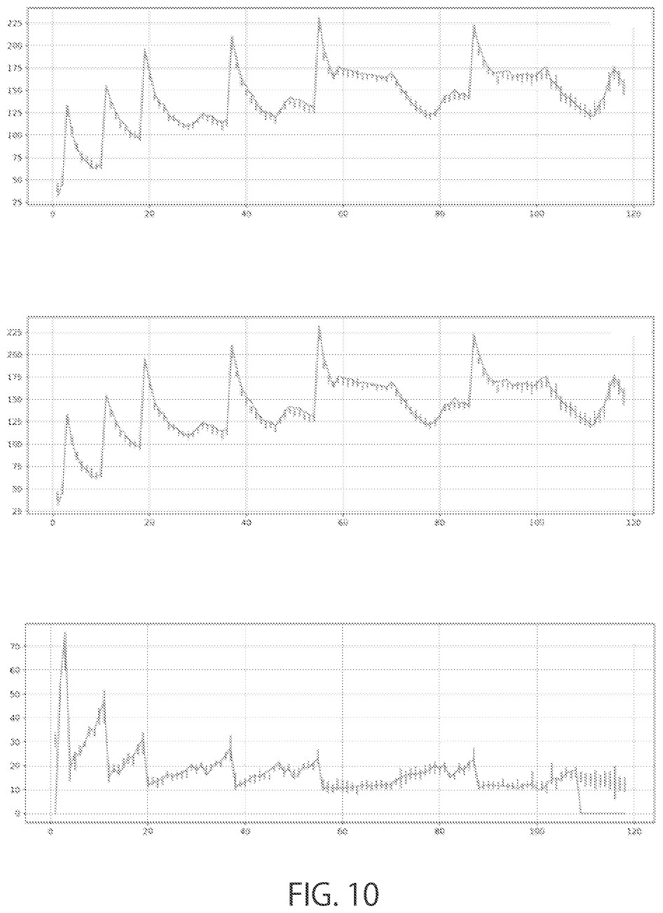

FIG. 10 illustrates some examples in which the feature extracted by the encoder 142 is decoded by the decoder 144. A solid line indicates the value of the teacher data, and a line having variance with respect to the atomic number indicates the output value of the decoder 144. The variation indicates the output value input into the decoder 144 while having variance with respect to the feature vector based on the feature output by the encoder 142 and the variance.

The graphs indicate examples of the covalent radius using the method of Pyykko, the van der Waals radius using UFF, and the second ionization energy in the descending order. The horizontal axis represents the atomic number, and the vertical axis is shown with a unit suitable for the examples.

The graph of the covalent radius shows that good values are output with respect to the teacher data.

It is shown that good values are output with respect to the teacher data also in the van der Waals radius and the second ionization energy. From an atomic number exceeding about 100, the value deviates, because it is the value which cannot be acquired as the teacher data at present and thus training is performed without the teacher data. Therefore, the variation in data increases, but a value at a certain level is output. Further, as explained above, it is shown that the second ionization energy of a hydrogen atom does not exist, but is output as an interpolated value.

It is shown that the use of the teacher data for the output from the decoder 144 makes it possible to accurately acquire the feature amount in the latent space in the encoder 142 as in the above.

(Structure Feature Extractor 18)

Next, the training of the second network and the third network of the structure feature extractor 18 will be explained.

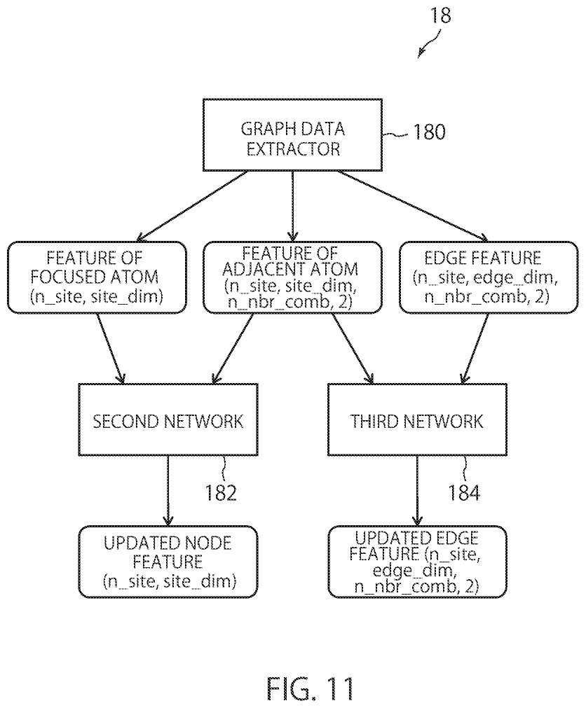

FIG. 11 is a diagram of extracted portions relating to the neural network of the structure feature extractor 18. The structure feature extractor 18 in this embodiment includes a graph data extractor 180, a second network 182, and a third network 184.

The graph data extractor 180 extracts the graph data such as the node feature and the edge feature from the data on the input structure of the molecule or the like. This extraction does not need to be trained when it is executed by the method on a rule base capable of inverse transformation.

However, a neural network may be used for the extraction of the graph data, in which case the neural network can be trained as a network including the second network 182, the third network 184, and the fourth network of the physical property value predictor 20 as well.

The second network 182, when receiving input of the feature of the focused atom (node feature) and the feature of the neighboring atom which are output from the graph data extractor 180, updates and outputs the node feature. For this update, the second network 182 may be formed of, for example, a neural network which applies the activation function, pooling, and batch normalization in order while separating the convolution layer, the batch normalization, the gate and the other data to transform the tensor from the (n_site, edge_dim, n_nbr_comb, 2) dimensions to the tensor of the (n_site, site_dim, n_nbr_comb, 1) dimensions, then applies the activation function, pooling, and batch normalization in order while separating the convolution layer, the batch normalization, the gate and the other data to transform it from the (n_site, site_dim, n_nbr_comb, 1) dimensions to the (n_site, site_dim, 1, 1) dimensions, and finally calculates the sum of the input node feature and the output to update the node feature via the activation function.

The third network 184, when receiving input of the feature of the neighboring atom and the edge feature which are output from the graph data extractor 180, updates and outputs the edge feature. For this update, the third network 184 may be formed of, for example, a neural network which applies the activation function, pooling, and batch normalization in order while separating the convolution layer, the batch normalization, the gate and the other data to perform transformation, then applies the activation function, pooling, and batch normalization in order while separating the convolution layer, the batch normalization, the gate and the other data to perform transformation, and finally calculates the sum of the input edge feature and the output to update the edge feature via the activation function. Regarding the edge feature, for example, the tensor of the same (n_site, site_dim, n_nbr_comb, 2) dimensions as that of the input is output.

The neural network formed as in the above can execute the error backward propagation from the output to the input because the processing in each layer is differentiable processing. Note that the above network composition is illustrated as an example, but not limited to this, and is a composition which can appropriately update the node feature to the one appropriately reflecting the feature of the neighboring atom, and may be any composition as long as it is a composition in which the operation of each layer is substantially differentiable. Substantially differentiable means the case of being approximately differentiable in addition to the case of being differentiable.

The error calculator 24 calculates an error based on the updated node feature propagated backward by the parameter updater 26 from the physical property value predictor 20 and the updated node feature output from the second network 182. The parameter updater 26 updates the parameter of the second network 182 using the error.

Similarly, the error calculator 24 calculates an error based on the updated edge feature propagated backward by the parameter updater 26 from the physical property value predictor 20 and the updated edge feature output from the third network 184. The parameter updater 26 updates the parameter of the third network 184 using the error.

As in the above, the neural networks included in the structure feature extractor 18 are subjected to training together with the training of the parameter of the neural network provided in the physical property value predictor 20.

(Physical Property Value Predictor 20)

The fourth network provided in the physical property value predictor 20 outputs the physical property value when receiving input of the updated node feature and the updated edge feature which are output from the structure feature extractor 18. The fourth network includes, for example, the structure of MLP or the like.

The fourth network can be trained by the same method as that for training the ordinary MLP or the like. The loss to be used is, for example, a mean absolute error (MAE), a mean square error (MSE) or the like. This error is propagated backward to the input of the structure feature extractor 18 to execute the training of the second network, the third network, and the fourth network as explained above.

The fourth network may be in a different form depending on the physical property value desired to be acquired (output). In other words, the output values of the second network, the third network, and the fourth network may be different ones, based on the physical property values desired to be found. Therefore, the fourth network may be made into an appropriate form or may be trained based on the physical property value desired to be acquired.

In this case, for the parameters of the second network and the third network, parameters which have already been trained or optimized for finding the other physical property values may be used as initial values. Further, a plurality of physical property values desired to be output as the fourth network may be set, in which case the training may be executed simultaneously using a plurality of physical property values as the teacher data.

As another example, the first network may also be trained by the backward propagation to the atom feature acquirer 14. Further, the first network is not trained in combination with the other networks from the beginning of the training to the fourth network but may be subjected to transfer learning by being trained in advance by the above-explained training method (for example, Variational Encoder Decoder using Reparametrization trick) of the atom feature acquirer 14 and then performing the backward propagation from the fourth network through the third network and the second network to the first network. This makes it possible to easily obtain the inferring device capable of obtaining the inference result desired to be found.

Note that the inferring device 1 including the neural network thus obtained is capable of the backward propagation from the output to the input. In other words, it is possible to differentiate the output data by a variable of the input. Thus, it is possible to know, for example, how the physical property value output from the fourth network changes by changing the input coordinates the atom. For example, in the case where the physical property value of the output is potential, position differentiation is force acting on each atom. The use of this also makes it possible to perform optimization of minimizing the energy of the structure of the input inferring object.

The training of each of the above-explained neural networks is performed as above in detail, and a generally known training method may be used as the whole training. For example, any of the loss function, the batch normalization, the training end condition, the activation function, the optimization technique, and the learning technique such as batch learning, mini batch learning, and online learning may be used as long as it is an appropriate one.

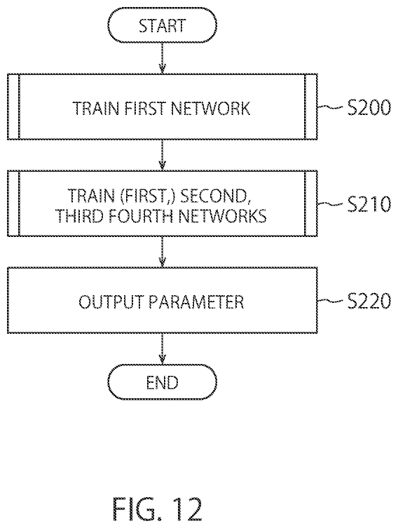

FIG. 12 is a flowchart illustrating the whole training processing.

The training device 2 first trains the first network (S200).

Subsequently, the training device 2 trains the second network, the third network, and the fourth network (S210). Note that the training device 2 may train also the first network as explained above at this timing.

When the training is finished, the training device 2 outputs the parameter of each of the trained networks via the output 22. Here, the output of the parameter is the concept including the output to the inside such as storing the parameter into the storage 12 in the training device 2 or the like together with outputting the parameter to the outside of the training device 2.

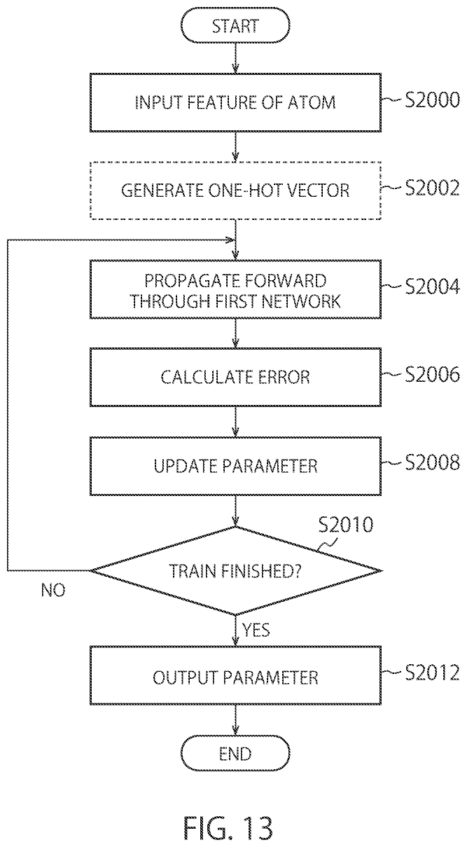

FIG. 13 is a flowchart illustrating the processing of the training of the first network (S200 in FIG. 12).

First, the training device 2 accepts input of data to be used for training via the input 10 (S2000). The input data is stored, for example, in the storage 12 as needed. The data required for training of the first network is a vector corresponding to an atom, information required for generating the one-hot vector in this embodiment, and an amount indicating the property of an atom corresponding to the above atom (for example, the substance amount of the atom). The amount indicating the property of the atom is, for example, the one indicated in FIG. 9. Besides, the one-hot vector itself corresponding to the atom may be input.

Next, the training device 2 generates a one-hot vector (S2002). This processing is not essential if the one-hot vector is input at S2000. In the other cases, the one-hot vector corresponding to the atom is generated based on the information to be transformed to the one-hot vector such as a proton number.

Next, the training device 2 propagates forward the generated or input one-hot vector to the neural network illustrated in FIG. 8 (S2004). The one-hot vector corresponding to the atom is transformed to a physical property value via the encoder 142 and the decoder 144.

Next, the error calculator 24 calculates an error between the physical property value output from the decoder 144 and the physical property value acquired from the chronological scientific tables or the like (S2006).

Next, the parameter updater 26 propagates backward the calculated error to update the parameter (S2008). The error backward propagation is executed up to the one-hot vector, namely, the input of the encoder.

Next, the parameter updater 26 determines whether the training is finished (S2010). This determination is made by a predetermined training end condition, for example, the end of a predetermined number of epochs, the securement of a predetermined accuracy, or the like. Note that the training may be, but not limited to, batch learning or mini batch learning.

When the training is not finished (S2010: NO), the processing from S2004 to S2008 is repeated. In the case of the mini batch learning, the processing may be repeated while changing the data to be used.

When the training is finished (S2010: YES), the training device 2 outputs the parameter via the output 22 (S2012) and finishes the processing. Note that the output may be the parameter relating to the encoder 142, namely, the parameter relating to the first network 146, or may be output together with the parameter of the decoder 144. The first network transforms the one-hot vector having the dimensions of 10² order to the vector indicating the feature in the latent space of, for example, 16 dimensions.

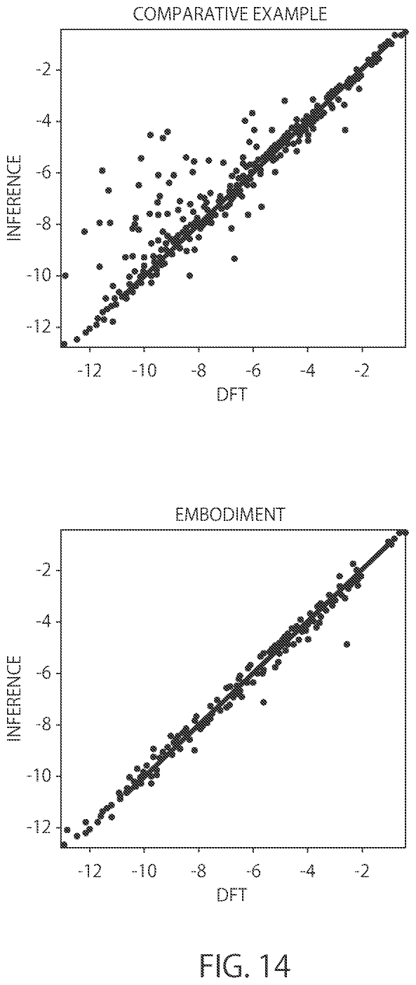

FIG. 14 is a chart illustrating the inference result of the energy of the molecule or the like by the structure feature extractor 18 and the physical property value predictor 20 trained using the output from the first network according to this embodiment as the input, and the inference result of the energy of the molecule or the like by the structure feature extractor 18 and the physical property value predictor 20 according to this embodiment trained using the output relating to the atom feature in a comparative example, (CGCNN: Crystal Graph Convolutional Networks, https://arxiv.org/abs/1710.10324v2) as the input.

The left graph is according to the comparative example and the right graph is according to the first network of this embodiment. These graphs indicate the values obtained by DFT on the horizontal axes and the values estimated by the respective methods on the vertical axes. In short, it is ideal that all of values exist on the diagonal line from the lower left toward the upper right, and more variation indicates lower accuracy.

These graphs show that the physical property value with less variation from the diagonal line and with higher accuracy can be output, namely, the more accurate feature of the atom (vector in the latent space) can be acquired as compared with the comparative example. The respective MAEs are 0.031 according to this embodiment and 0.045 according to the comparative example.

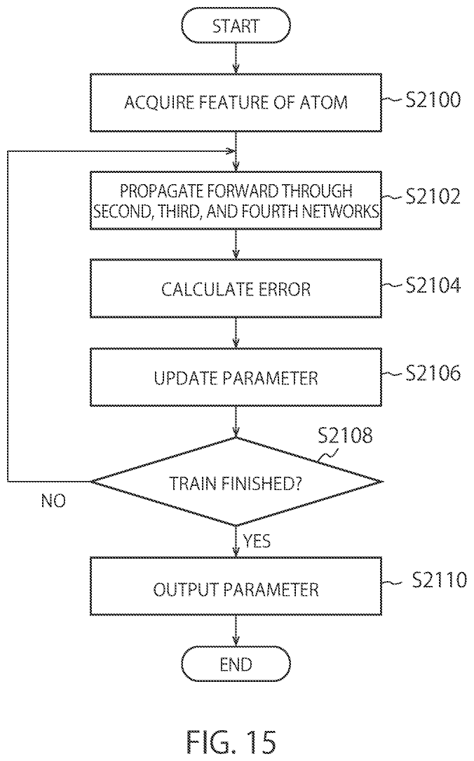

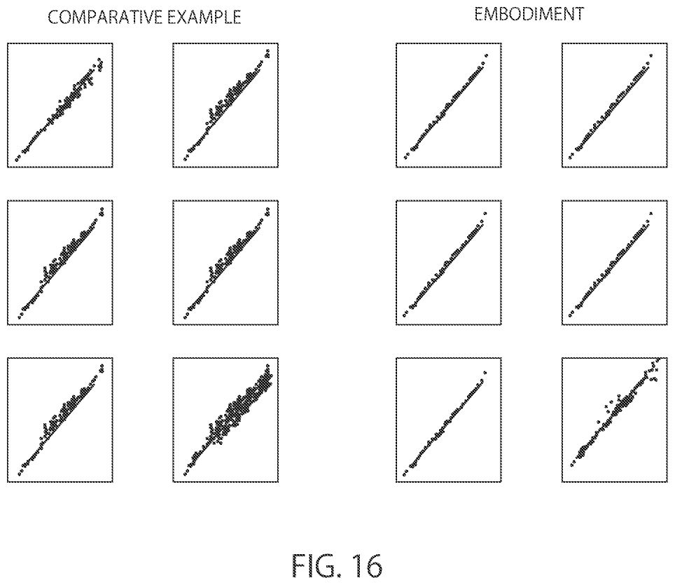

Next, an example of processing for the training of the second network to the fourth network will be explained. FIG. 15 is a flowchart illustrating an example of the processing of the training of the second network, the third network, and the fourth network (S210 in FIG. 12).

First, the training device 2 acquires the feature of the atom (S2100). This acquisition may be performed every time by the first network, or may be performed by storing the feature of each atom inferred by the first network in advance in the storage 12 and reading out the data.

Next, the training device 2 transforms the feature of the atom to the graph data via the graph data extractor 180 of the structure feature extractor 18, and inputs the graph data into the second network and the third network. The updated node feature and the updated edge feature acquired by forward propagation are input into the fourth network after being processed when necessary, thereby propagating forward them through the fourth network (S2102).

Next, the error calculator 24 calculates the error between the output from the fourth network and the teacher data (S2104).

Next, the parameter updater 26 propagates backward the error calculated by the error calculator 24 to update the parameter (S2106).

Next, the parameter updater 26 determines whether the training is finished (S2108), and when the training is not finished (S2108: NO), repeats the processing at S2102 to S2106, whereas when the training is finished (S2108: YES), outputs the optimized parameter (S2110) and finishes the processing.

In the case of training the first network using the transfer learning, the processing in FIG. 15 is performed after the processing in FIG. 13. When performing the processing in FIG. 15, the data to be acquired at S2100 is the data on the one-hot vector. Then, at S2102, the data is propagated forward through the first network, the second network, the third network, and the fourth network. Necessary processing, for example, processing executed by the input information composer 16 is also appropriately executed. Then, the processing at S2104 and S2106 is executed to optimize the parameter. For updating on the input side, the one-hot vector and the error propagated backward are used. As explained above, by learning again the first network, the vector in the latent space acquired in the first network can be optimized based on the physical property value desired to be finally acquired.

FIG. 16 illustrates examples in which the value inferred according to this embodiment and the value inferred by the above-explained comparative example are obtained for some physical property values. The left side indicates the values in the comparative example and the right side indicates the values according to this embodiment. The horizontal axis and the vertical axis are the same as those in FIG. 14.

The chart shows that the variation in the values according to this embodiment is small as compared with those in the comparative example, and that the physical property values close to the result of DFT can be inferred.

As in the above, according to the training device 2 according to this embodiment, it is possible to acquire the feature of the property (physical property value) as the atom, as the vector of a low dimension, and to perform inference with high accuracy of the physical property value of the molecule or the like by the machine learning by transforming the acquired feature of the atom to the graph data including the angular information and inputting it into the neural network.

In this training, the architectures of the feature extraction and the physical property value prediction are common, so that when the kinds of atoms are increased, the amount of the learning data can be reduced. Further, since the atom coordinates and the neighboring atom coordinates of each atom only need to be included in the input data, those coordinates can be applied according to various forms of the molecule, crystal and so on.

The inferring device 1 trained by the training device 2 can infer at high speed the physical property value such as energy or the like of a system using an arbitrary atom arrangement such as molecule, crystal, molecule and molecule, molecule and crystal, crystal interface or the like as input. Further, the physical property value can be position-differentiated, and therefore the force or the like acting on each atom can be easily calculated. For example, for energy, various physical property value calculation using the first-principles calculation requires enormous calculation time so far, but this energy calculation can be performed at high speed by forward propagation through the trained network.

As a result of this, for example, the structure can be optimized to minimize the energy, and the property calculation of various substances can be increased in speed based on the energy or the differentiated force by cooperation with a simulation tool. Further, for example, regarding the molecule or the like with changed atom arrangement, energy can be inferred at high speed only by changing the coordinates of the input and inputting the changed coordinates into the inferring device 1 without performing complicated energy calculation again. As a result of this, it is possible to easily perform material search in a wide range by simulation.

Some or all of each device (the inference device 1 or the training device 2) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

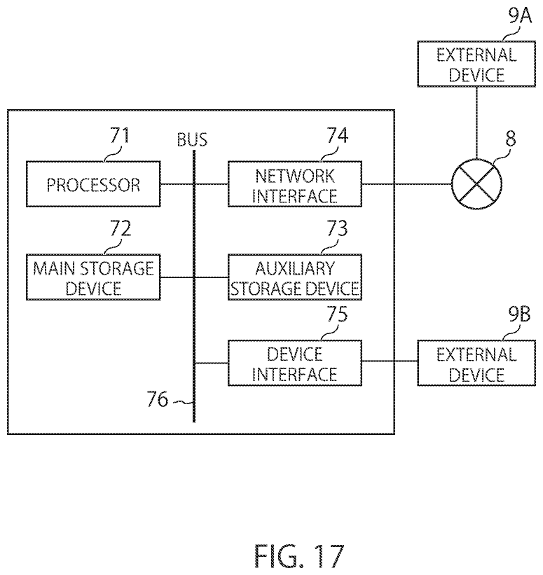

FIG. 17 is a block diagram illustrating an example of a hardware configuration of each device (the inference device 1 or the training device 2) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 17 is provided with each component one by one but may be provided with a plurality of the same components. Although one computer 7 is illustrated in FIG. 17, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the inference device 1 or the training device 2) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the inference device 1 or the training device 2) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the inference device 1 or the training device 2) in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the inference device 1 or the training device 2) in the above embodiments may be enabled by the main storage device 72 or the auxiliary storage device 73 or may be implemented by a built-in memory built into the processor 71. For example, the storages 12 in the above embodiments may be implemented in the main storage device 72 or the auxiliary storage device 73.

At least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8.

The external device 9A may include, for example, a camera, a motion capture, a device which is outputted, an external sensor, or input device. An external storage device (an external memory), for example, a network storage may be included as the external device 9A. Further, the external device 9A may be a device having each device (the inferring device 1 or the training device 2) which described in some of above-mentioned embodiments. And, the computer 7 may receive and/or transmit to external of the computer 7 a part of or all the processing results via the communication network 8.

The device interface 75 is an interface such as, for example, a USB that directly connects to the external device 9B. The external device 9B may be an external storage medium or a storage device (memory). The storage unit 12 in the above-mentioned embodiment may be realized by the external device 9B.

The external device 9B may be, as an example, an output device. The output device may be, for example, a display device such as, for example, an LCD (Liquid Crystal Display), CRT (Cathode Ray Tube), PDP (Plasma Display Panel) or an organic EL (Electro Luminescence) panel, a speaker, a personal computer, a tablet terminal, or a smart phone, but not limited to these. Further, the external device 9B may be an inputting device. The inputting device may include, for example, a device such as a keyboard, a mouse, a touch panel, or a microphone, and provide the inputted information to the computer 7 inputted by these devices.

The external device 9B may be, for example, an HDD storage.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “based on data,” “according to data,” “in accordance with data,” unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrically connection/coupling,” “communicatively connection/coupling,” “operatively connection/coupling,” “physically connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including claims), when a plurality of hardware performs a predetermined process, the respective hardware may cooperate to perform the predetermined process, or some hardware may perform all the predetermined process. That is, when it is described that “one or more hardware perform a first process and the one or more hardware perform a second process,” or the like, is used, the hardware that perform the first process and the hardware that perform the second process may be the same hardware, or may be the different hardware.

In the present specification (including the claims), when a plurality of processors perform a plurality of processes, each processor among the plurality of processors may perform only a part of the plurality of processes, may perform all of the plurality of processes, and may not perform any of the plurality of processes in some cases.

In the present specification (including the claims), when a plurality of storage devices (memories) store data, an individual storage device among the plurality of storage devices may store only a part of the data or may store the entire data, or may not store any date in some cases.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

For example, in the above embodiments, the characteristic value is inferred using the feature of the atom, and information such as the temperature of the system, the pressure, the charge in the whole system, the spin of the whole system and so on may be taken into consideration. Such information may be input, for example, as a super node connected to each node. In this case, a neural network capable of receiving input of the super node is formed, thereby making it possible to output an energy value or the like in consideration of the information on the temperature or the like.

### APPENDIX

Each of the above embodiments can be illustrated as follows when using, for example, a program.

(1) A program including, when executed by one or more processors:

inputting a vector relating to an atom into a first network which extracts a feature of the atom in a latent space from the vector; and

inferring the feature of the atom in the latent space through the first network.

(2) A program including, when executed by one or more processors:

composing a structure of atoms of an object based on input coordinates of the atoms, features of the atoms, and a boundary condition;

acquiring a distance between atoms and an angle formed by three atoms based on the structure; and

updating a node feature and an edge feature, using the feature of the atom as the node feature and using the distance and the angle as the edge feature, to infer the node feature and the edge feature.

(3) A program including, when executed by one or more processors:

inputting a vector indicating a property of an atom included in an object into the first network according to any one of claim 1 to claim 7, to extract the feature of the atom in the latent space;

composing a structure of atoms of the object based on coordinates of the atoms, extracted features of the atoms in the latent space, and a boundary condition;

inputting a node feature based on the feature of the atom and the structure into the second network according to any one of claim 10 to claim 12, to acquire the updated node feature;

inputting an edge feature based on the feature of the atom and the structure into the third network according to any one of claim 13 to claim 16, to acquire the updated edge feature; and

inputting the acquired updated node feature and updated edge feature into a fourth network which infers a physical property value from a feature of a node and a feature of an edge, to infer the physical property value of the object.

(4) A program including, when executed by one or more processors:

inputting a vector relating to an atom into a first network which extracts a feature of the atom in a latent space from the vector relating to the atom;

inputting the feature of the atom in the latent space into a decoder which, when receiving input of the feature of the atom in the latent space, outputs a physical property value of the atom, to infer a characteristic value of the atom;

the one or more processors calculating an error between the inferred characteristic value of the atom and teacher data;

propagating backward the calculated error to update the first network and the decoder; and

outputting a parameter of the first network.

(5) A program including, when executed by one or more processors:

composing a structure of atoms of an object based on input coordinates of the atoms, features of the atoms, and a boundary condition;

acquiring a distance between atoms and an angle formed by three atoms based on the structure;

inputting information based on the features of the atoms, the distance and the angle into a second network which acquires an updated node feature using the feature of the atom as a node feature and into a third network which acquires an updated edge feature using the distance and the angle as an edge feature;

calculating an error based on the updated node feature and the updated edge feature; and

propagating backward the calculated error to update the second network and the third network.

(6) A program including, when executed by one or more processors:

inputting a vector indicating a property of an atom included in an object into a first network which extracts a feature of the atom in a latent space from the vector relating to the atom, to extract the feature of the atom in the latent space;

composing a structure of atoms of the object based on coordinates of the atoms, extracted features of the atoms in the latent space, and a boundary condition;

acquiring a distance between atoms and an angle formed by three atoms based on the structure;

inputting a node feature based on the feature of the atom and the structure into a second network which acquires an updated node feature using the feature of the atom as the node feature, to acquire the updated node feature;

inputting an edge feature based on the feature of the atom and the structure into a third network which acquires an updated edge feature using the distance and the angle as the edge feature, to acquire the updated edge feature;

inputting the acquired updated node feature and updated edge feature into a fourth network which infers a physical property value from a feature of a node and a feature of an edge, to infer the physical property value of the object;

calculating an error from the inferred physical property value of the object and teacher data; and

propagating backward the calculated error through the fourth network, the third network, the second network, and the first network to update the fourth network, the third network, the second network, and the first network.

(7) Each of the programs according to (1) to (6) may be stored in a non-transitory computer-readable medium, and the one or more processors may execute the methods according to (1) to (6) by reading one or more of the programs according to (1) to (6) stored in the non-transitory computer-readable medium.

1. An inferring device comprising:
one or more memories; and
one or more processors, wherein:
the one or more processors are configured to:
input a vector relating to an atom into a first network which extracts a feature of the atom in a latent space from the vector relating to the atom; and
infer the feature of the atom in the latent space through the first network.

2. The inferring device according to claim 1, wherein
the vector relating to the atom includes a symbol representing the atom or information similar to the symbol, or includes information acquired based on the symbol representing the atom or the information similar to the symbol.

3. The inferring device according to claim 1, wherein
the first network is composed of a neural network having an output dimension lower than an input dimension.

4. The inferring device according to claim 1, wherein
the first network is a model trained by Variational Encoder Decoder.

5. The inferring device according to claim 1, wherein
the first network is a model trained using a physical property value of the atom as teacher data.

6. The inferring device according to claim 3, wherein
the first network is a neural network composing an encoder of the trained model.

7. The inferring device according to claim 1, wherein:
the vector relating to the atom is expressed by a one-hot vector; and
the one or more processors are configured to:
when receiving input of information relating to the atom, transform the information to the one-hot vector; and
input the transformed one-hot vector into the first network.

8. The inferring device according to claim 1, wherein
the one or more processors are configured to further
infer a physical property value of a substance being an inferring object including the inferred atom, based on the inferred feature of the atom.

9. An inferring device comprising:
one or more memories; and
one or more processors, wherein:
the one or more processors are configured to:
compose a structure of an inferring object based on input coordinates of atoms, features of the atoms, and a boundary condition;
acquire a distance between atoms and an angle formed by three atoms based on the structure; and
update a node feature and an edge feature, using the feature of the atom as the node feature and using the distance and the angle as the edge feature, to infer an updated node feature and an updated edge feature, respectively.

10. The inferring device according to claim 9, wherein
the one or more processors are configured to:
extract a focused atom from the atoms included in the structure;
search for a predetermined number or less of atoms existing in a predetermined range from the focused atom as neighboring atom candidates;
select two neighboring atoms from the neighboring atom candidates;
calculate a distance between each of the neighboring atoms and the focused atom based on the coordinates; and
calculate the angle formed between the two neighboring atoms and the focused atom using the focused atom as a vertex based on the coordinates.

11. The inferring device according to claim 10, wherein
the one or more processors are configured to
input the node feature into a second network which, when receiving input of the node feature of the focused atom and the node features of the neighboring atoms, outputs the updated node feature, to acquire the updated node feature.

12. The inferring device according to claim 11, wherein
the second network is composed including a neural network capable of processing graph data.

13. The inferring device according to claim 9, wherein
the one or more processors are configured to
input the edge feature into a third network which, when receiving input of the edge feature, outputs the updated edge feature, to acquire the updated edge feature.

14. The inferring device according to claim 13, wherein
the third network is composed including a neural network capable of processing graph data.

15. The inferring device according to claim 13, wherein
the one or more processors are configured to
when acquiring different features with respect to a same edge from the third network, average the different features with respect to the same edge to regard the averaged feature as the updated edge feature.

16. The inferring device according to claim 9, wherein
the feature of the atom is obtained from the inferring device.

17. The inferring device according to claim 16, wherein
the feature of the atom included in the inferring object acquired through the first network is acquired in advance and stored in the one or more memories.

18. The inferring device according to claim 9, wherein
the one or more processors are configured to further
infer the physical property value of the inferring object based on the updated node feature and the updated edge feature.

19. The inferring device according to claim 18, wherein
the one or more processors are configured to
input the acquired updated node feature and updated edge feature into a fourth network which infers the physical property from a feature of a node and a feature of an edge, to infer the physical property value of the inferring object.

20. A training device comprising:
one or more memories; and
one or more processors, wherein:
the one or more processors are configured to:
input a vector relating to an atom into a first network which extracts a feature of the atom in a latent space from the vector relating to the atom;
input the feature of the atom in the latent space into a decoder which, when receiving input of the feature of the atom in the latent space, outputs a physical property value of the atom, to infer a characteristic value of the atom;
calculate an error between the inferred characteristic value of the atom and teacher data;
propagate backward the calculated error to update the first network and the decoder; and
output a parameter of the first network.
